(19) United States
(12) Patent Application Publication
(10) Pub. No.: US 2023/0285535 A1
(43) Pub. Date: Sep. 14, 2023

(54) Recombinant Pseudorabies Virus and Vaccine Composition thereof

(71) Applicant: NOVO BIOTECH CORP., Shaoxing (CN)

(72) Inventors: Qiang ZHANG, Shaoxing (CN); Hong QIAN, Shaoxing (CN); Youqiang WU, Shaoxing (CN)

(73) Assignee: NOVO BIOTECH CORP., Shaoxing (CN)

(21) Appl. No.: 18/095,016

(22) Filed: Jan. 10, 2023

Related U.S. Application Data
(63) Continuation of application No. PCT/CN2021/104472, filed on Jul. 5, 2021.

(30) Foreign Application Priority Data
Jul. 10, 2020 (CN) 202010661997.0

(51) Int. Cl. A61K 39/12; A61P 37/04

(52) U.S. Cl. CPC A61K 39/12; A61P 37/04

(57) ABSTRACT

The present application provides a recombinant Pseudorabies virus whose genome contains nucleotide sequences coding the capsid protein P72, the accessory protein B602L and the exterior envelope protein CD2V derived from African swine fever virus. This recombinant Pseudorabies virus can be used to prepare live virus vector vaccine to effectively treat or prevent African swine fever and overcome defects of the existing inactivated vaccines and attenuated live vaccines.

### CROSS-REFERENCE TO RELATED APPLICATION

The present application claims the priority of the Chinese patent application No. CN202010661997.0 submitted on Jul. 10, 2020. The contents disclosed in the prior application are incorporated into the present application by reference in its entirety.

### REFERENCE TO SEQUENCE LISTING

The Sequence Listing submitted concurrently herewith under 37 CFR § 1.821 in a computer readable form (CRF) via EFS-Web as file name SEQUENCE LISTING.xml is incorporated herein by reference. The electronic copy of the Sequence Listing was created on Jun. 3, 2023, with a file size of 14 KB.

### TECHNICAL FIELD

The present application belongs to the technical field of genetic engineering vaccine, more specifically, relates to a recombinant Pseudorabies virus rPRV and an African swine fever vaccine prepared by this rPRV virus.

### TECHNICAL BACKGROUND

African swine fever (ASF) is an acute, strong and highly contactable contagion caused by African swine fever virus (ASFV), which is characterized by its short pathogenetic process, being the most acute and a death rate up to 100% for an acute infection. Its clinic syndromes include fever (up to 40˜42° C.), a quickened heartbeat, breathing difficulties, a slight cough, serous or mucosity purulent secretion in eyes and nose, cyanosis of the skin, obvious bleeding in lymph nodes, kidney and gastrointestinal mucosa. Clinic syndromes of African swine fever are similar to those of the classic swine fever and World Organization for Animal Health (OIE) lists African swine fever as a notifiable communicable animal epidemic disease. China also lists it as a type of animal epidemic disease. This disease was firstly discovered in 1921 in Kenya, Africa. Since 2007, African swine fever occurs, spreads and outbreaks in many countries around the world, especially in Russia and its surrounding areas. In 2018, ASF epidemic has occurred in our country, which causes enormous direct and indirect economic losses.

ASFV is a double strand DNA virus with an envelope and its genome is an unimolecular linear double strand DNA and has a whole length about 170 kb-190 kb, where the center thereof has a conserved region about 125 kb and both ends thereof are variable regions and containing terminal inverting and repeating sequences. ASFV genome codes a hypothetical membrane protein, a secretory protein, an enzyme involving the nucleotide and nucleic acid metabolism (DNA reparation) and protein modification and its entire genome contains 151 ORF coding 150-200 proteins.

ASFV can be detected from blood, tissue fluid, internal organs and other excrements of infected pigs. ASFV virus in blood preserved in a low temperature anechoic chamber can survive for six years and can survive for several weeks under room temperature. ASFV virus can be destroyed only when blood infected by the virus is heated at 55° C. for 30 minutes or is heated at 60° C. for 10 minutes and can be destroyed by many fat solvents and disinfectors.

The vaccine is an important means to prevent and treat ASFV. The existing ASFV vaccine candidates mainly include inactivated vaccines and attenuated live vaccines. The inactivated vaccines refer to the vaccines obtained by deactivating ASFV through physical or chemical methods, wherein the deactivation deprives their infection ability but maintain their antigenicity. So far, inactivated ASF vaccine prepared by multiple traditional methods, including the alveolar macrophages inoculated with virus and the inactivated vaccine through homogenate preparation from an infected spleen tissue, all failed to provide effective immune protection against the challenge.

The attenuated live vaccine refers to the vaccine prepared by a live virus of the attenuated ASFV strain. The attenuated ASFV strains include passage attenuated strains, natural attenuated strains and recombinant attenuated strains. The passage attenuated strains refers to the strains obtained due to the gradually decreasing pathogenicity of ASFV during the passage procedure of cell lines such as cells derived from a porcine bone marrow, Vero and COS-1. For example, the isolated strain ASFV-G is cultivated in Vero cells for passage culture and the virulence of the virus gradually weakens and completely disappears upon the 110^{th} generations. However, domestic pigs inoculated with the passage attenuated ASFV-G strain fail to obtain the corresponding protection so as to resist the attack of parent virus. Besides, reports have shown that side effects such as pneumonia, miscarriage and death as well as ASFV chronic infection clinic syndromes occur in domestic pigs inoculated with the passage attenuated ASFV-G strain. The natural attenuated strains refers to the naturally existing attenuated strains, such as ASFV OURT88/3 or NH/P68 strains. However, many side effects such as pneumonia, miscarriage and death can occur to animals immuned with the natural attenuated strains. For example, 25%˜47% of pigs immuned with NH/P68 strains present chronic infection; pigs immuned with OURT88/3 can have syndromes such as fever and swelling joint etc.

The recombinant attenuated strains refer to the strains obtained by virus functional genes knockout, virus virulence genes knockout or immune suppressor genes knockout through the molecular biology method. The recombinant attenuated strains decrease the virus virulence or strengthens and the immune response of the organism to virus, and can be used to develop genetically engineered attenuated live vaccine with better safety and better effect than traditional low virulent vaccine. ASFV virulence genes and immune suppressor genes including TK(K196R), 9GL(B119L), CD2V(EP402R), DP148R, NL(DP71L), UK(DP96R) and multigene family 360 and 505 (MGF 360/505) as well as genes associated with immune escapeA238L, A179L, A224L, DP71L, MGF360/505, I329L, K205R, D96R, DP148R, A276R, D96R and EP153R etc, have been reported. In 2019, Zhang Yanyan et al from Military Medical Sciences knocked out the domestically separated MGF and CD2V sequences on the domestically separated SY18 virulent strain through the genetic engineering method. Preliminary experimental results show that attack from the parent strain SY18 can be 100% resisted, wherein pigs in the control group are all dead and pigs from the immunized group have clinic syndromes such as fever, which means the safety in a long term administration still needs further verification. At the end of 2019, American researchers discovered that low virulent vaccines prepared from the deletion of I177L genes can provide 100% protection for pigs and the immunized pigs also do not spread virus to unimmunized pigs. But its safety still needs further verification.

The live vector vaccines refer to ones that clone genes coding proteins of the pathogene into the live virus vector and then express the protein within immuned animals so as to induce the immune response to the proteins. Compared with other vaccines, advantages of the live vector vaccine lie in (1) the capability of automatically infecting tissues or cells and increasing the efficiency of exogenous genes entering into cells; (2) the vector's inherent adjuvant effect enabling them to induce the generation of cytokines and chemokines; (3) the capability of the majority thereof to include immune response in a long term.

Pseudorabies virus (PRV) belongs to the a Herpes virus subfamily of Herpetoviridae and it has a wide host range including the livestock such as pigs, dogs, cows, goats and wild animals, but it does not infect humans. It has strong proliferation ability in host cells and is easy for amplification, which makes it a vaccine vector with strong development potential due to the facts that it has clear genetic background and strong genetic stability, contains many non-essential genes for virus duplication, has a large volume of exogenous genes (40 kb), presents a stable recombinant PRV genetic characters and its exogenous genes cannot be easily lost.

The prior art reports some live vector vaccines. For example, CN108504686A and CN108504687A respectively provide the recombinant adenovirus vector expressing EP153R and EP402R genes of ASFV. Argilaguet J M et al constructed BacMam-sHAPQ based on rod-shaped virus vectors, wherein the immunized pigs can induce the specific T cell response, part of the pigs can resist the attack from the homologous and sublethal virulent strain and a large volume of IFN-γ secreting type T cells are detected in pig blood after 17 days' challenge. Lokhandwala S et al respectively inserted p32, p54, pp62 and p72 genes into replication-deficient adenovirus vector by using adenovirus vectors and pigs immuned with recombinant adenovirus virus are shown to be capable of inducing the generation of African swine fever virus specific antibody of high quality, cell immune response and cytotoxic T lymphocyte reaction; in 2016, Lokhandwala S et al respectively recombined A151R, B119L, B602L, EP402RAPRR, B438L, K205R and A104R into a adenovirus vector, wherein strong IgG response specific to African swine fever antigen and IFN-y are generated after pigs are immuned by the recombinant adenovirus virus added with adjuvants. In 2017, Loperamadrid J et al screened out 5 antigens of ASFV through the Vaxign system, wherein p72, p54, p12 antigens expressed by human embryo kidney 293 (HEK) cells and 3 MVA vector antigens (B646L, EP153R and EP402R) are immunized by the primary and booster immunization, the humoral immune response can be strengthened through inoculation of ASFV proteins purified by HEK cells while the cell immunization is rather weak and MVA vector antigen can facilitate the cell immunization to generate IFN-γ without any reports on results of protection against the challenge. In 2020, Lynnette C et al adopted the adenovirus virus and poxvirus as the vector to express 8 ASFV genes including B602L, B646L(p72), CP204L(p30), E183L(p54), E199L, EP153R, F317L and MGF505-5R, wherein these genes of a high immunization dosage can provide a complete protection for pigs against African swine fever and part of them have serious side effect, still resulting in the occurrence of African swine fever syndromes, which makes it unsafe and discourages its scaled production and application due to its large immunization dosage and massive genes in need of expression. Therefore, there are no reports that the existing live vector vaccine can provide effective protection for immunized pigs against African swine fever and this is because African swine fever virus has a complicated structure and presents as a multiple layer structure, which makes a single component can hardly be effectively protective.

### SUMMARY OF THE INVENTION

Pseudorabies virus (PRV) as a live vector of African swine fever virus is well studied in order to construct the recombinant attenuated strain so as to prepare live vector vaccine used for treating and preventing African swine fever. By means of massive screening immunogen proteins, it is surprisingly discovered that PRV simultaneously expresses the exterior envelope CD2V, the outer capsid protein p72 and the accessory protein B602L component. Especially, the organism can be effectively activated to generate a protective antibody when soluble p72 accounts for over 20% of the total amount of the capsid protein p72, which can effectively and safely protect pigs from attack of lethal dosage. This immunogen mixture contains the outermost envelope CD2V and the outermost capsid protein p72 and it simultaneously expresses the accessory protein B602L in order to ensure the simultaneous accurate folding and expression of the capsid protein p72, which lays the foundation for a novel recombinant live vector subunit vaccine. Specifically, the present application contains the following technical solutions.

A recombinant Pseudorabies virus (rPRV), wherein its genome contains the following exogenous genes:

A nucleotide sequence A coding the capsid protein p72 derived from African swine fever virus or variants thereof, the variants are constructed by substitution, deletion or addition of one or more amino acid residues in the amino acid sequence of the capsid protein p72 and have the function of the capsid protein p72; preferably, the amino acid sequence of the capsid protein p72 variants has a homology over 90%, preferably has a homology over 95%, more preferably has a homology over 98%, more preferably has a homology over 99% to the capsid protein p72;

A nucleotide sequence B coding a accessory protein used to prompt the accurate expression and folding the capsid protein p72 or variants thereof, the variants are constructed by substitution, deletion or addition of amino acid residues in the amino acid sequence of the accessory protein and have the function of prompting the accurate expression and folding the capsid protein p72 or variants thereof; preferably, the amino acid sequence of this accessory protein variants has a homology over 90%, preferably has a homology over 95%, more preferably has a homology over 98%, more preferably has a homology over 99% to the accessory protein;

A nucleotide sequence C coding an exterior envelope protein CD2V derived from African swine fever virus or variants thereof, the variants are constructed by substitution, deletion or addition of one or more amino acid residues in the amino acid sequence of the exterior envelope protein CD2V and have the function of the exterior envelope protein CD2V; preferably, the amino acid sequence of the exterior envelope protein CD2V variants has a homology over 90%, preferably has a homology over 95%, more preferably has a homology over 98%, more preferably has a homology over 99% to the exterior envelope protein CD2V.

Preferably, the above accessory protein can be an accessory protein B602L derived from African swine fever virus.

The above exogenous genes can merely contain the nucleotide sequence A, the nucleotide sequence B and the nucleotide sequence C. Namely, the recombinant Pseudorabies virus merely expresses these 3 heterologous proteins of the capsid protein p72, the accessory protein B602L and the exterior envelope protein CD2V but does not express other heterologous proteins.

The above recombinant Pseudorabies virus (rPRV) is suitable for replicating and expressing the nucleotide sequence A, the nucleotide sequence B and the nucleotide sequence C within cells, wherein the cells are chosen from cells used for virus proliferation, such as mammalian cells, bird cells or insect cells. For example, examples of available cells include a alveolar macrophage, cells derived from porcine bone marrow, Vero cells, COS-1 cells, human embryo kidney 293 (HEK) cells, a chicken Embryo fibroblast (CEF), Chinese hamster ovary cells (CHO), baby hamster kidney cells (BHK), African green monkey kidney cells (VERO), cervical cancer cells (HELA), perC6 cells, sf9 cells etc.

Preferably, the solubility of the capsid protein p72 accounts for over 20% of the total amount of the capsid protein p72 in the above cells. Researches discover that the soluble capsid protein p72 has the function of an immunogen, or the immune effect is poor even if the capsid protein p72 is expressed but is not soluble.

Replication of rPRV within these cells can massively prepare African swine fever vaccine.

In one preferable embodiment, the above nucleotide sequence A has a homology≥90%, preferably has a homology≥95%, more preferably has a homology≥98%, more preferably has a homology≥99% to SEQ ID NO:1; the nucleotide sequence B has a homology≥90%, preferably has a homology≥95%, more preferably has a homology≥98%, more preferably has a homology≥99% to SEQ ID NO:2; the nucleotide sequence C has a homology≥90%, preferably has a homology≥95%, more preferably has a homology≥98%, more preferably has a homology≥99% to SEQ ID NO:3.

In another optional embodiment, the genome of the above recombinant Pseudorabies virus can also contain following exogenous genes:

A nucleotide sequence D coding a capsid protein p49 derived from African swine fever virus or variants thereof, the capsid protein p49 variants are constructed by substitution, deletion or addition of one or more amino acid residues in the amino acid sequence and have the function of the capsid protein p49; preferably, the amino acid sequence of the capsid protein p49 variants has a homology over 90%, preferably has a homology over 95%, more preferably has a homology over 98% to the capsid protein p49.

In other words, apart from the nucleotide sequence A, the nucleotide sequence B and the nucleotide sequence C, the genome of the above recombinant Pseudorabies virus can further contains exogenous genes chosen from the nucleotide sequence D and a nucleotide sequence E. Namely, apart from these 3 heterologous proteins of the capsid protein p72, the accessory protein B602L and the exterior envelope protein CD2V, the recombinant Pseudorabies virus can further express heterologous proteins chosen from the capsid protein p49.

Preferably, a deletion and/or replacement occur in at least one of the non-essential replication region in the genome of the above recombinant Pseudorabies virus.

The above non-essential replication region can be chosen from one or more than two of gC, gE, gG, gI, gM, TK, RR, PK coding region of Pseudorabies virus.

For example, a deletion occurs in gE and/or gG coding the non-essential replication region in the PRV genome.

Preferably, in the above recombinant Pseudorabies virus, the nucleotide sequence A, the nucleotide sequence B and the nucleotide sequence C are respectively located in the non-essential replication region of the genome. These exogenous genes can be located in one identical non-essential replication region or can be located in a different non-essential replication region.

In the above recombinant Pseudorabies virus, the expression cassette of the capsid protein p72 genes can be CMV-p72-bGH, wherein CMV is CMV promoter, the capsid protein p72 is the target gene and bGH is bovine growth hormone terminator. For the convenience of detection, 5 terminal of the target gene of the capsid protein p72 is fused with a flag label.

The expression cassette of the above B602L genes can be SV40-B602L-hGH, wherein SV40 is SV40 promoter, B602L is the target gene and hGH is human growth hormone terminator. For the convenience of detection, 3 terminal of the target gene B602L is fused with HA label.

The expression cassette of the above exterior envelope protein CD2V genes can be EF1α-CD2V-HSV-TK, wherein EF1α is EF1α promoter, the exterior envelope protein CD2V is the target gene and HSV-TK is the herpes virus TK gene terminator. For the convenience of detection, 3 terminal of the target gene of the exterior envelope protein CD2V is fused with His label.

The second aspect of the present application provides a method of constructing the above recombinant Pseudorabies virus and the method comprises the following steps:
(1) Substituting the coding region TK genes in the genome of Pseudorabies viral strain PRV-BAC (HL) with a codon-optimized gene expression cassette expressing the capsid protein p72 derived from African swine fever virus to obtain PRV-BAC-p72-ΔTK;
(2) Inserting the codon-optimized gene expression cassette of the accessory protein B602L derived from African swine fever virus into the capsid protein p72 genes to obtain PRV-BAC-p72-B602L-ΔTK;
(3) Substituting the coding region gG genes of PRV-BAC-p72-B602L-ΔTK obtained in step (2) with the codon-optimized gene expression cassette of the exterior envelope protein CD2V derived from African swine fever virus to obtain PRV-BAC-p72-B602L-CD2V-ΔTK-ΔgG;
(4) Transfecting BHK-21 cells with PRV-BAC-p72-B602L-CD2V-ΔTK-ΔgG obtained in step (3) to save and obtain the recombinant Pseudorabies virus that simultaneously expresses the capsid protein p72, the accessory protein B602L and the exterior envelope protein CD2V proteins and is named as rPRV-p72-B602L-CD2V-ΔTK-ΔgG.

For the convenience of optimization and expression, nucleotides of genes coding the capsid protein p72, the accessory protein B602L, the exterior envelope protein CD2V proteins can also be replaced without any influences on the amino acids, which can be understood by a person skilled in the art.

Specifically, step (1) can be realized by the following steps:
a. inserting the codon-optimized the capsid protein p72 genes derived from African swine fever virus into the vector pEE12.4-kan to obtain pEE12.4-p72-kan;
b. performing PCR amplification on a proper primer by using pEE12.4-p72-kan as the template. For example, when the coding sequence of the capsid protein p72 is SEQ ID NO:1, the primer is SEQ ID NO:4 and SEQ ID NO:5.
c. transferring the target fragment obtained in step b into PRV-BAC/GS1783 electroporated competent cells (such as ones in a colibacillus GS1783 competent state) and producing PRV-BAC-p72-kan-ΔTK by Red/ET recombination; the second recombination deletes kan genes in PRV-BAC-p72-kan-ΔTK to obtain PRV-BAC-p72-ΔTK bacterial strain.

Similarly, the accessory protein B602L and the exterior envelope protein CD2V can be respectively inserted into the capsid protein p72 and then can be inserted into gG site with the same recombination method. For example, the codon-optimized the accessory protein B602L genes derived from African swine fever virus are inserted into pSV40-kan to obtain Psv40-B602L-kan and then are performed with steps such as amplification, subsequent ligation and transformation; inserting the codon-optimized the exterior envelope protein CD2V genes derived from African swine fever virus into pEF1α-kan to obtain pEF1α-CD2V-kan and then are performed with steps such as amplification, subsequent ligation and transformation to finally obtain PRV-BAC-p72-B602L-CD2V-ΔTK-ΔgG bacterial strain.

The third aspect of the present application provides an application of the above recombinant Pseudorabies virus in preparing vaccines used for preventing and treating African swine fever.

The above recombinant Pseudorabies virus can be a live vector subunit vaccine.

The fourth aspect of the present application provides African swine fever vaccine or an immunological composition (or called as immunogenic composition), which at least contains the above recombinant Pseudorabies virus as the immunogen.

In the Pseudorabies virus composition of the present application, following components are also contained: Pseudorabies virus whose the genome contains the nucleotide sequence A coding the above capsid protein p72 or variants thereof and the nucleotide sequence B coding the above accessory protein such as B602L; and/or Pseudorabies virus whose genome contains the nucleotide sequence C coding the above exterior envelope protein CD2V or variants thereof.

Preferably, the above African swine fever vaccine or immunological composition can also contain veterinarily acceptable vectors, excipients or adjuvants.

As a live virus vector vaccine, the above recombinant Pseudorabies virus can be replicated within a pig body and can express the immunological antigen of African swine fever (namely the capsid protein p72 and the exterior envelope protein CD2V derived from African swine fever virus).

When the recombinant Pseudorabies virus constructed in the present application acts as a the live virus vector vaccine to immune a mouse, it can detects the capsid protein p72, the accessory protein B602L, the exterior envelope protein CD2V antibodies from the mouse body, which shows that antibodies against the capsid protein p72 and the exterior envelope protein CD2V derived from ASFV are successfully activated in animals, lays a foundation for the development of PRV-ASFV recombinant live vector vaccine. This rPRV live virus vaccine can overcome defects of the inactivated vaccine and attenuated live vaccine in the prior art and shows excellent prospect for their industrial development and application.

### SPECIFICATION OF DRAWINGS

FIG. 1 is the schematic diagram of the eukaryotic expression plasmid pEE12.4-kan.

FIG. 2 is the agarose gel electrophoresis photo of the recombinant PRV-BAC after the enzyme digestion verification, wherein M: the DL15000 Marker; 1, 2, 3: the recombinant PRV-BAC-CMV-p72-bGH-ΔTK; 4: the PRV-BAC (HL) control.

FIG. 3 is SDS-PAGE gel electrophoresis photograph photo of the capsid protein p72 expression after the western-blot verification, wherein M: the protein Marker; 1, 2, 3, 4: the supernatant of different screened strains lysate cells containing rPRV-p72--B602L-CD2V-ΔTK-ΔgG; 5: the supernatant of negative control cell lysate.

FIG. 4 is SDS-PAGE gel electrophoresis photograph of CD2V expression after the western-blot verification, wherein M: the protein Marker; 1: the positive protein control containing His label, being about 55kd; 2, 3, 4, 5: the supernatant of different screened strains lysate cells containing rPRV-p72--B602L-CD2V-ΔTK-ΔgG.

### SPECIFIC EMBODIMENTS

The present application adopts Pseudorabies virus as the vector to express African swine fever immunogen, namely the capsid protein p72, the accessory protein B602L and the exterior envelope protein CD2V derived from ASFV, and prepares the recombinant vaccine used for preventing and treating African swine fever through cultivation and replication thereof within proper cells.

The amino acid sequence of the capsid protein p72 derived from ASFV is SEQ ID NO:6; the amino acid sequence of the accessory protein B602L derived from ASFV is SEQ ID NO:7; the amino acid sequence of the exterior envelope protein CD2V derived from ASFV is SEQ ID NO:8.

The recombinant Pseudorabies virus constructed in the present application can be replicated within a pig body and can express the immunological antigen of African swine fever (namely the capsid protein p72 and the exterior envelope protein CD2V derived from African swine fever virus).

African swine fever virus is of multiple layer structure, wherein the heterologous protein CD2V is located in the outmost layer and the capsid protein p72 is located in the second layer. Researches show that the accurate expression and accurate folding the capsid protein p72 for the maintenance of specific spatial structure of the capsid protein p72 is the key factor for the recombinant Pseudorabies virus to generate immunogenicity to African swine fever. In the recombinant Pseudorabies virus, the accessory protein B602L as the chaperonin can prompt the accurate expression and accurate folding the capsid protein p72.

Further researches and clinic experiments show that the recombinant Pseudorabies virus also allows the expression of other proteins such as the capsid protein p49 derived from African swine fever virus (ASFV), but the species and amount of proteins allowed for loading are limited, or the accurate folding the capsid protein p72 will be influenced and the immunogenicity of the recombinant Pseudorabies virus will be seriously compromised. For example, the loading some proteins such as p12, p14 (E120R), pE248R, p22, p32, p54 will generate adverse influences. Therefore, it is an optimal choice to merely load nucleotide sequences A-C coding the capsid protein p72, the accessory protein B602L and the exterior envelope protein CD2V in the genome of Pseudorabies virus, such as SEQ ID NOs: 1-3. On the other hand, the decrease on the amount of the heterologous protein facilitates the construction steps of the recombinant Pseudorabies virus, which is also advantageous. Seen from this point, it is also a preferable solution to merely load nucleotide sequences A and B coding the capsid protein p72 and the exterior envelope protein CD2V in the genome of Pseudorabies virus, such as SEQ ID NOs: 1-2.

In the present text, for the convenience of description, sometimes a certain protein such as the capsid protein p72 is called interchangeably with the name of its coding genes (DNA) and a person skilled in the art should understand that they represent different substances under different description situations. A person skilled in the art can easily understand their meanings according to the language environment and the context. For example, the capsid protein p72 refers to the protein for the description of the capsid protein function or species and refers to genes coding this capsid protein p72 when it is described as a gene.

In the present application, the term “recombined Pseudorabies virus”, “recombinant Pseudorabies virus”, “recombinant virus”, “recombinant PRV” and “rPRV” represent the same meanings and can be used interchangeably. The term “African swine fever vaccine”, “PRV-ASFV vaccine” and “recombinant vaccine” represent the same meanings and all refer to the recombinant African swine fever subunit vaccine prepared by adopting the Pseudorabies virus as the vector and can be used interchangeably.

When constructing the recombinant Pseudorabies virus, the codon optimization can be performed to the specific expression host or vector such as Pseudorabies virus and the host cell for the sake of realizing the optimal expression of heterologous proteins in different expression host or vectors. The codon optimization is a technique to optimize the protein expression in organisms by increasing translational efficiency of interested genes. Different organisms normally show special preference to one of some codons coding the same amino acids due to the mutation tendency and the natural selection. For example, in host cells under fast growth, optimized codons reflect the composition of its respective genome tRNA database. Therefore, in host cells under fast growth, the low frequency codon for an amino acid can be replaced with the high frequency codon for the same amino acid. Therefore, the expression of optimized DNA sequences can be improved in host cells under fast growth. Gene sequences of SEQ ID NO:1-3 provided in the present text are codon-optimized nucleotide sequences, but expression genes of the capsid protein p72, the accessory protein B602L and the exterior envelope protein CD2V in the present application are not limited thereby.

The term “host cell” covers constructs and all cells related to the construction process of vectors in the present application, including but not limited to bacteria, yeast cells, mammalian cells.

The recombinant Pseudorabies virus needs to be saved in a host cell for replication and amplification to express the heterologous protein. The term “rescue” refers to the process of allowing non-proliferatively infected virus to complete replication and proliferation and produce offspring virus through certain methods. Major methods of saving a virus include co-cultivation and co-infection.

A person skilled in the art can easily understand that African swine fever vaccine or the immunological composition in the present application can be veterinarily acceptable vectors, excipients or adjuvants.

The term “immunological composition” can be called as “immunogenic composition” and refers to the composition containing at least one antigens and the antigen induces immunity response in hosts administrated with the immunogenic composition.

The term “veterinarily acceptable vector” includes but not limited to a solvent, a dispersing media, a coating agent, a stabilizer, a thinner, a preservative, an antibacterial and antifungal agent, an isotonic agent, a adjuvant, an immune attack agent and the combination thereof.

These vectors, for example, consist of a stable salt, an emulsifier, a solubilizer or an osmotic pressure regulator, a suspending agent, a thickener and a redox agent of maintaining the physiological redox potential. Preferable adjuvants include an aluminum salt, a microemulsion, a lipid particle and/or the oligonucleotide used to increase the immune response.

The term “vector” refers to a diluent such as water, salt, dextrose, ethanol, glycerin and phosphate buffered saline (PBS), excipients or mediums capable of administrating compositions. The vector as a solid composition in the pharmaceutical composition can include an adhesive such as microcrystalline cellulose and polyvinylpyrrolidone (povidone or polyvinylpyrrolidone), tragacanth gum, gelatin, starch, lactose or lactose monohydrate; a decompositing agent such as alginic acid, cornstarch and the like; a lubricant or surfactant, such as magnesium stearate or sodium lauryl sulfonate; a glidant such as a silica gel; a sweetening agent such as a sucrose or a saccharin; a stabilizer such as but not limited to the alkali metal salt of the albumin and ethylenediaminetetraacetic acid.

The term “adjuvant” refers to non-specific immune enhancer, which can enhance the immune response of organisms to antigens or change the immune response type when injected together with the antigen or injected into the organism in advance. African swine fever vaccine or immunological composition in the present application can or cannot include adjuvants.

Proper adjuvants can be chosen from an aluminiferous adjuvant (such as an aluminum hydroxide, an aluminum phosphate, an alum), a lipopolysaccharide, a Freund's Complete adjuvant, a Freund's Incomplete adjuvant, a CpG oligonucleotide, a mineral gum, an aluminum hydroxide, a surfactant, a lysolated lecithin, a Plulanick polyol, a polycationic or oil emulsion such as water in oil or oil in water or combination thereof. Of course, selections on the adjuvant depend on purposive applications. For example, the virulence may depend on subjected organisms and can range from being non-toxic to highly toxic.

The vaccine or the immunological composition in the present application can be used for treating or preventing African swine fever virus (ASFV) infection.

The term “treating or preventing” normally involves the administration of animals in need (mainly a pig) with the effective vaccine or the immunological composition in the present application. The term “treating” refers to the administration of the vaccine or the immunological composition in an effective dosage after an animal individual or at least some animals in a group are infected with ASFV and these animals have already shown clinic syndromes caused by ASFV infection or ones related thereto. The term “preventing” refers to the administration of the vaccine or the immunological composition in an effective dosage before an animal is not infected by ASFV or does not show any clinic syndromes caused by ASFV infection or ones related thereto.

The term “effective dosage” includes but is not limited to the amount of antigen inducing or capable of inducing immune response in individuals. The effect amount can decrease the morbidity of ASFV infection in small animals or reduce the severe degree of clinic syndromes of ASFV infection.

The term “clinic syndrome” refers to the signs of ASFV infection. Examples of the clinic syndromes of ASFV infection include but not limited to fever (up to 40-42° C.), rapid heartbeat, difficulty breathing, slight cough, serous or mucopurulent discharge from the eyes and nose, cyanosis of the skin, significant bleeding the lymph nodes, kidneys, gastrointestinal mucosa.

In the present application, the “effective dosage” can be 10⁵-10⁹ pfu, preferably 10⁶-10⁸ pfu, more preferably 10⁷ pfu. In some embodiments, the vaccine or the immunological composition in the present application are compositions in a liquid form. In some embodiments, the volume of the vaccine or the immunological composition in the present application is 0.5-5 mL, preferably 1-4 mL, more preferably 1.5-3 mL, such as 1.5 mL, 2 mL, 2.5 mL or 3 mL

Now the present application will be explained in detail in combination with specific embodiments. It should be understanding that the following embodiments are merely for the sake of describing the present application rather than limiting the scope of the present application.

Unless otherwise specified, the present text involves the addition amount, the content and the concentration of multiple substances, wherein the percentage contents all refer to the weight percentage content.

### EMBODIMENTS

Materials and methods

Gene synthesis, primer synthesis and sequencing in the embodiments are all completed by Nanking GenScript Biotech Corporation.

The molecular biology experiments in the embodiments include plasmid construction, enzyme digestion, ligation, competent cell preparation and transformation and medium preparation etc, which can be performed mainly by referring to Molecular Cloning A Laboratory Manual (third edition), edited by J. Joseph Sambrook, D. W. Russell (US), translated by Huang Peitang et al, Science Press, Beijing, 2002. Specific experimental conditions can be ascertained through simple experiments when necessary.

PCR amplification experiments can be performed based on reaction conditions or kits provided by plasmid or DNA template providers, which can be adjusted through simple experiments when necessary.

LB culture medium: 10g/L tryptone, 5 g/L yeast extract, 10 g/L sodium chloride, high temperature and high pressure sterilization at 121° C. of pH7.2 for 20 minutes.

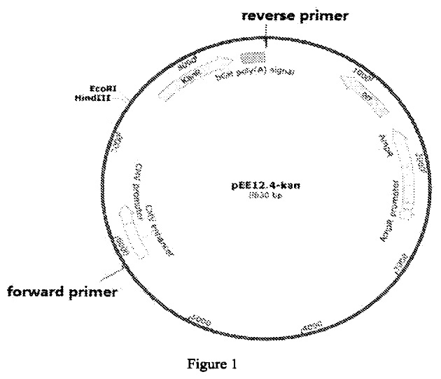

The recombinant PRV-BAC (HL strain) GS1783 bacterial strain, eukaryotic expression plasmid pEE12.4-kan (this plasmid is transformed from pEE12.4 (purchased from Shanghai Linyuan Biotech Co Ltd), which is added with kan resistance gene and is integrated into the artificial chromosome of bacteria for screening and verification) (the plasmid profile is shown as FIG. 1) and BHK-21 cells are preserved in Novo Biotech and any units and individuals can obtain these cells and plasmid to verify the present application but cannot be used for other purposes including the development and exploitation, the scientific research and education without the consent of Novo Biotech.

Wherein, the construction method for the artificial chromosome (BAC) of PRV-BAC (HL) bacteria (colibacillus) can briefed as follows: firstly, a piggery in Shaoxing, Zhejiang obtains the strain PRV (HL) by separation and US2 and U6 gene fragment of the PRV (HL) act as the homologous arms and are sequentially cloned into pUC19 vector to construct pUC19-US2-US6; miniF fragments carrying BAC elements and marked with green fluorescence (GFP) into pUC19-US2-US6 to construct the transfer vector miniF-US2-US6; co-transfecting BHK-21 cells with the PRV (HL) genome and the transfer vector miniF-US2-US6 and screening the bacteriophage plaque containing green fluorescent protein to obtain the PRV recombinant virus PRV-BAC (HL) carrying the BAC vector, followed by extracting the genome of the recombinant virus PRV-BAC (HL) genome, electroporating GS1783 competent cells, coating LB agar plate containing 50μg/ml kanamycin and 34 μg/ml chloromycetin, cultivating it overnight at 32° C. , selecting the monoclonal colony in LB liquid culture medium containing 50 μg/ml kanamycin and 34 μg/ml, cultivating it overnight at 32° C. to obtain PRV-BAC (HL) GS1783 bacterial strain.

### Embodiment 1 Selection on Subunit Proteins of African Swine Fever Virus and Optimization of Gene Sequences Thereof

1.1 Selection on Proteins of the African Swine Fever Virus

The constitutive protein CD2V derived from African swine fever virus is the glycosylated protein located in the exterior envelope and coded by EP402R genes. A transmembrane domain is predicted to exist in 207-229aa and researches have shown that the exterior envelope protein CD2V can interact with the erythrocyte and presents very important function during the process epidemic diffusion and lymphocyte damage. A person skilled in the art commonly knows that it is the exterior envelope protein CD2V fragment located in the extracellular region part that have the function of interacting with the host cell, which makes the ideal protective antigen. For the convenience of the exterior envelope protein CD2V expression, the fragments deleted with the transmembrane domain are chosen, likewise, for the convenience of the exterior envelope protein CD2V expression, a person skilled in the art can select the exterior envelope protein CD2V extracellular region fragment, the exterior envelope protein CD2V extracellular region and other fragments such fused fragments of Fc or Cd3 or fragments of the exterior envelope protein CD2V deleted with th transmembrane domain (207-229aa) deleted etc. The constitutive protein p72 derived from African swine fever virus is a stretch of polypeptide coding by the accessory protein B646L genes. The dynamics of the capsid protein p72 synthesis shows that this protein is translated in the later period of infection and accounts for about 32% of the total protein amount, which is the main protein of icosahedral structure of the virus and is not reported in Pseudorabies although the capsid protein p72 is reported to be expressed in multiple systems. Researchers have shown that the constitutive protein B602L of African swine fever virus is a stretch of polypeptide coded by the accessory protein B602L genes and the accessory protein B602L can prompt the accurate folding the capsid protein p72. The expression of the capsid protein p72 will obviously be decreased if the accessory protein B602L is lacked. Although this protein is not the constitutive protein of the virus, great changes will occur to the assembly of the virus if this protein is lacked, which will finally result in the failure of the accurate assembly into the virus particle. Therefore, these 3 proteins are simultaneously chosen to be expressed in the Pseudorabies vector.

1.2 Gene Synthesis

The epidemic African swine fever strain subtype reported in China, 2018 is adopted as the template by referring to Georgia 2007/1 complete genome sequence (GenBank: FR682468.1) and the codons of the B646L nucleotide sequence coding the capsid protein p72 of African swine fever are optimized to obtain OPTI-p72 sequence as shown by SEQ ID NO: 1. For the convenience of subsequent construction and detection, HindIII enzyme digestion sites are added in 5′ terminal of the genes and Flag label sequences are added therein, EcoRI enzyme digestion sites are added in 3′ terminal. The synthesized sequence is subcloned to pUC57 and named as pUC57-OPTI-p72; codons of the B602L nucleotide are optimized to obtain OPTI-B602L sequence as shown by SEQ ID NO:2. For the convenience of subsequent detection, HA label is added in C terminal; codons of the exterior envelope protein CD2V nucleotide are optimized to obtain OPTI-CD2V as shown by SEQ ID NO:3. For the convenience of detection, His label is added in C terminal and the synthesized sequence is subcloned to pUC57 and is named as pUC57-OPTI-CD2V. The sequence synthesis is completed by Nanking GenScript Biotech Corporation.

### Embodiment 2 Construction of the Intermediate Vector pEE12.4-p72-kan

2.1 1.5 ml EP tube is marked and the plasmid pEE12.4-kan and pUC57-OPTI-p72 obtained in Embodiment 1 are respectively performed with double enzyme digestion by means of Hind III and EcoRI, wherein the enzyme digestion system (50 ul) is shown as follows.

| Name of sampled component | volume (μL) |
|---|---|
| dd H₂O | supplemented to 50 |
| 10×buffer | 5 |
| DNA sample | volume when the mass is 2 μg |
| HindIII | 2.5 |
| EcoRI | 2.5 |

2.2 1.5 mL EP tube in step 2.1 is placed in a thermostat water bath with a proper temperature of the corresponding enzyme for 2-3 hours' waterbath. Gel extraction of the double enzyme digestion products is performed as follows: the above double enzyme digestion system is taken out for agarose gel electrophoresis to extract DNA fragments therein.

2.3 Ligation Reaction

(1) several 1.5 mL EP tubes are provided and marked, which are placed on an EP tube shelf for later use.

(2)1.5 mL EP tubes are sampled as the following table and mixed uniformly.

| Name of sampled | | |
|---|---|---|
| component | experiment group(μL) | control group(μL) |
| dd H₂O | l | 6 |
| 10× T4 ligated buffer | 1 | 1 |
| target fragment | 6 | — |
| pEE12.4-kan | 2 | 2 |
| T4 ligase | 1 | 1 |

(3) After sampling according to the table in step (2) is completed, each 10 μl reaction system is placed in a low temperature coolant circulating machine at 16° C. for 10-16 hours' waterbath;

(4) EP tubes in step (3) are taken out and placed in a 65° C. water bath for 15 minutes' waterbath;

(5) EP tubes in step (4) are placed at 4° C. for preservation.

2.4 Transformation Reaction

(1) 10 μL ligation reaction solution is added quickly into 100 μL colibacillus JM109 competent cells (purchased from Takara), which is blew and beaten, mix uniformly and performed with ice bath for 30 minutes;

(2) the sample tubes are taken out to be placed in a 42° C. waterbath for 100 seconds, which is then immediately performed with ice bath for 2 minutes;

(3) sample tubes are taken out, which are added with 600 μL liquid LB culture medium on a bechtop and placed on a 37° C. constant temperature shaker at 220 rpm/min for 1 hour's cultivation;

(4) coating plate: sample tubes in a step (3) are taken out, which are centrifuged at 8,000 rpm/min for 2 minutes at room temperature and removed with 600 μL supernatant liquid. The thallus in the tube bottom of remained supernatant is resuspended and the resuspended bacteria solution is placed into the center of the corresponding transformed plate, followed by evenly spreading the bacteria solution in the center of the transformed plate with a fungus coating stick.

(5) the transformed plate in the transformation step (4) is placed upright in a biochemical constant temperature incubator for 1 hour's cultivation at 37° C. and the transformed plate is then placed upside down for 15 hours' cultivation;

(6) Observing the transformation results.

2.5 Plasmid Extraction and Double Enzyme Digestion Identification

2.5.1 Plasmid Extraction (by Use of the Kit DP6943, OMEGA)

(1) monoclone is taken out from the transformed plate with a 10 μL pipette tip into 5 mL LB liquid culture medium containing Ampicin resistance, which is shaked at 37° C., 220 rpm/min overnight;

(2) transferring the bacteria solution into a 1.5 mL EP tube, which is centrifuged at 12,000 rpm/min for 2 minutes under room temperature and abandoned with supernatant;

(3) 250 μL plasmid extraction reagent P1 buffer is added into a EP tube in step (2) and the thallus is thoroughly suspended;

(4) 250 μL P2 buffer is added into the solution in step (3), the centrifuge tube is immediately and mildly put upside down and mixed uniformly for 5-10 times, which is standing for 2-4 minutes;

(5) 350 μL P3 buffer is added into the solution in step (4), the centrifuge tube is immediately and wildly put upside down and mixed uniformly for 5-10 times, which is standing for 2-4 minutes;

(6) the solution in step (5) is centrifuged at 14,000 rpm/min for 10 minutes under room temperature;

(7) the supernatant solution in step (6) is transferred to the center of a adsorption column, which is centrifuged at 12,000 rpm/min for 30 seconds under room temperature and the liquid in the collection tube is abandoned;

(8) 500 μL buffer DW1 is added into the adsorption column, which is centrifuged at 12,000 rpm/min for 30 seconds under room temperature and the liquid in the collection tube is abandoned;

(9)500 μL cleaning solution is added into the center of the adsorption column, which is centrifuged at 12,000 rpm/min for 30 seconds under room temperature, the liquid in the collection tube is abandoned and the procedure is repeated for once;

(10) the empty adsorption column is centrifuged at 12,000 rpm for 2 minutes under room temperature.

(11) a adsorption column is put into a 1.5 mL clean centrifuge tube, 30 μL Elution buffer is added into the center of an adsorbed membrane, which is standing for 5 minutes under room temperature and centrifuged at 12,000 rpm for 2 minutes under room temperature. DNA solution in the tube is preserved.

2.5.2 Double Enzyme Digestion Identification

(1) 1.5 mL EP tube to be used are marked and sampled according to the followed table: a 20 μL reaction system

| Name of sampled component | volume (μL) |
|---|---|
| dd H₂O | supplemented to 20 μL |
| 10×buffer | 2 |
| DNA sample | volume when the mass is 1 μg |
| HindIII | 1 |
| EcoRI | 1 |

(2) 20 μL reaction system in the EP tube in step (1) is placed in a 37° C. thermostat water bath for 2 hours' water bath.

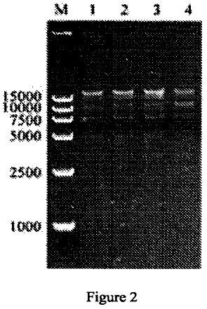

(3) samples in the double enzyme digestion system in step (2) is performed with agarose gel electrophoresis to check whether the size of the inserted fragment is right or not; the experiment results can refer to FIG. 2. The construction of the enzyme digestion is accurate.

(4) the clone with an accurate inserted fragment is sent to a sequencing company for sequencing. It is verified that the intermediate vector pEE12.4-p72-kan is constructed.

### Embodiment 3 Subcloning CMV-p72-bGH-kan Sequence

3.1 The Recombinant Sequence with CMV Promoter, the Capsid Protein p72 genes, I-sce I-kan and TK Homologous Arm is Amplified Through PCR

(1) adopting the pEE12.4-p72-kan constructed in Embodiment 2 as a template, whose forward and reverse primers are SEQ ID NO: 4 and SEQ ID NO:5.

For the convenience of recombination, the recombinant sequence with TK homologous arm in the PRV genome is amplified through PCR.

(2) 50 μL sampling system is shown by the following table:

| sampled component | volume (μL) |
|---|---|
| Q5 Mix | 25 |
| forward primer (10 μM) | 2.5 |
| reverse primer (10 μM) | 2.5 |
| pEE12.4p72-kan | 1 |
| ddH₂O | 19 |
| total volume | 50 |

PCR amplification procedure: 95° C. for 2 minutes; 95° C. for 30 seconds, 55° C. for 45 seconds, 72° C. for 1 minute, 30 circulations; 72° C. for 10 minutes; maintained at 8° C.

PCR products are performed with gel extraction, whose steps are as followed:
(1) sample collection EP tubes, adsorption columns and collection tubes are marked;
(2) weighing the mass of the marked empty EP tubes and recording values of the mass;
(3) the single target DNA band is carefully cut from the sepharose gel on a glue cutting machine and put into a clean 1.5 mL centrifuge tube;
(4) the 1.5 mL centrifuge tube in step (3) is added with 600 μL PC buffer, which is standing in a 50° C. waterbath for about 5 minutes, during which time the centrifuge tube is turned upside down mildly to ensure a complete dissolution of gommures;
(5) balancing the column: the adsorption column CB2 (the adsorption column is put into a collection tube in advance) is added with 500 μL equilibrium liquid BL, which is centrifuged at 12,000 rpm/min for 1 minute, the waste liquid in the collection tube is abandoned and the adsorption column CB2 is put back into the collection tube;
(6) the solution obtained in step (5) is added into the adsorption column CB2, which is standing for 2 minutes and centrifuged at 10,000 rpm/min for 30 seconds, the waste liquid in the collection tube is abandoned and the adsorption column CB2 is put into the collection tube;
(7) the adsorption column is added with 600 μL washing liquid PW buffer, which is standing for 3 minutes and centrifuged at 10,000 rpm/min for 30 seconds, the waste liquid in the collection tube is abandoned and the adsorption column CB2 is put into the collection tube;
(8) repeating the step (7);
(9) the empty adsorption column is centrifuged at 12,000 rpm/min for 2 minutes to remove the washing liquid to the great extent , the adsorption column is standing for 10 minutes under room temperature to get it completely dried;
(10) the adsorption column CB2 is put into a collection tue, 50 μL Elution buffer (preheated to 65° C.) is added by floating in the center of the adsorption membrane, which is standing for 3 minutes and centrifuged at 12,000 rpm/min for 2 minutes;
(11) the centrifuge tube in step (10) is taken out from a centrifuge, the adsorption column CB2 in the center is abandoned, the lid of the centrifuge tube covered, DNA samples in the centrifuge tube is preserved in the centrifuge tube;
(12) DNA samples in step (11) is standing at 4° C. for preservation, gel extraction DNA fragments by gel extraction is identified by the agarose gel electrophoresis.

3.2 Preparing Colibacillus GS1783 Competent Cells Electroporated with PRV-BAC(HL strain) GS1783

(1) GS1783 bacteria solution containing PRV-BAC (HL strain) preserved at-80° C. is streaked with a single colony in LB culture medium containing 1% chloromycetin (Chl) and then cultured at 32° C. for 16 hours, and the single monocolony is selected to be inoculated with 5 mL liquid LB (30 μg/m Chl), which is cultured at 32° C. for 12 hours overnight;

(2) GS1783 bacteria solution containing PRV-BAC(HL strain) is inoculated by 1 ml according to the ratio of 1:100 in a 100 ml LB conical flask, which is shaked at 220 rpm under 32° C. for 2-4 hours until the OD600 is between 0.5-0.7;

(3) the solution is quickly put into a 42° C. waterbath, which is shaked at 220 rpm for 15 minutes;

(4) the conical flask is put into an ice bath for 20 minutes and the prepared 10% glycerinum is cooled in advance;

(5) the bacteria solution in the conical flask in poured to a centrifuge cup and prepared in balance, which is centrifuged at 5000 rpm and 4° C. for 5 minutes and abandoned with supernatant;

(6) the solution is washed by adding 100 ml 10% glycerinum, which is shaked uniformly, centrifuged at 5000 rpm and 4° C. for 10 minutes and abandoned with supernatant;

(7) the step (6) is repeated two times, finally the supernatant is abandoned and about 2-3 ml of the bacterial solution is remained.

(8) the solution is blew and heat with Tips head, which is separated by 100 μl and into a 1.5 ml EP tube for preservation at −80° C. for later use.

3.3 Preparing PRV-BAC-CMV-p72-bGH-kan-ΔTK by Electroporation and the First Homologous Recombination

The subclonal product obtained by PCR in step 3.1 is electroporated into GS1783 competent cells in step 3.2. Specific steps are as follow:
(1) 100 ng PCR products in step 3.1 is added to recombinant electroporated competent cells in step 3.2;
(2) DNA/thallus mixture is transferred into an electroporation cup cooled in advance. The electroporation is performed at the condition of 15 kV/cm (1 mm electroporation cup is 1.5 kV) for 6.0 milliseconds;
(3) electroporated samples are transferred to a 1.5 ml EP tube and added with 1 ml nonreactive LB culture medium, which are cultured at 32° C. for 1-2 hours;
(4) after centrifuged at 6000 rpm for 1 minute, the product is coated on LB solid plate containing 30 μg/ml chloromycetin and 30 μg/ml kanamycin, which is cultured at 32° C. for 24 hours;
(5) positive clones are obtained through colony PCR.

3.4 PRV-BAC-CMV-p72-bGH-kan-ΔTK After the First Homologous Recombination by the Isopropyl Alcohol Precipitation Method

A single colony is selected in the accurate positive clones verified by PCR in step 3.3 and put into 6 ml LB culture medium containing 30 μg/ml Chl and 30 μg/ml kan, which is cultured at 32° C. for 24 hours;

(1) 5 mL bacteria solution is collected ,which is centrifuged at 5,000×g for 10 minutes under 4° C.;

(2) the culture medium is dumped or absorbed, which is then abandoned;

Note: in order to ensure that the culture medium is all abandoned, excess liquid on the wall is absorbed with a clean absorbent paper.

(3) adding 250 μl Solution I/RNaseA, which is performed with vortex and blew and beaten with a tip to resuspend the thallus and a complete suspension is crucial to obtain excellent yield;

Note: RNaseA must be added before the use of Solution I.

(4) adding 400 μL Solution II and the centrifuge tube is slightly and mildly overturned and rotated for 8-10 times until a clarified lysate is obtained; this step may need to be incubated for 2-3 minutes under room temperature and alternatively overturned and mixed uniformly with a total time not exceeding 5 minutes.

Note: vigorous agitation should be avoided to prevent the breaking DNA of chromosome and the decrease of plasmid purity. Solution II after use is covered with a lid tightly and is preserved under room temperature so as to avoid the reaction with CO₂ in the air;

(5) adding 200 μL N3 Buffer cooled in advance and slightly overturning the centrifuge tube for 20 times until the occurrence of flocculent precipitation, which is then put into ice bath for 10 minutes;

Note: a sufficient mixing is crucial to obtain a high yield. If the mixture looks viscous, brown or not mixed uniformly, it is mixed continuously until the solution is neutralized.

(6) the mixture is centrifuged at a full speed and 4° C. for 10 minutes to allow the cell debris to be completely precipitated;

(7) 700 μL supernatant is absorbed and put into a clean 1.5 ml centrifuge tube, which is then added with isopropanol cooled in advance to realize a final system concentration of 70%. The solution is slightly overturned for several times and performed with ice bath for 10 minutes;

(8) the solution is centrifuged at a full speed and 4° C. for 10 minutes to allow the plasmid to be completely precipitated;

(9) the supernatant is the abandoned and 700 μL 70% ethanol (fresh) is added, which is overturned upside down mildly to wash the precipitation and is centrifuged at 12000 rpm and 4° C. for 10 minutes;

(10) the step (9) is repeated;

(11) the supernatant is abandoned completely and is dried by strong wind on a super clean bench under room temperature for about 15 minutes;

(12) the precipitation is dissolved with 100 μl Elution Buffer (pre-heated at 65° C.);

(13) the plasmid concentration is detected with a Nano instrument (1000-2000 ng/μl is appropriate).

3.5 Verification of PRV-BAC-CMV-p72-bGH-kan-ΔTK After the First Homologous Recombination

The plasmid extracted in step 3.4 is verified with BamHI enzyme digestion and a negative control is provided at the same time. The enzyme digestion system is as follow:

| Name | System | |
|---|---|---|
| PRV-BAC plasmid | 5 | μg |
| BamH I^{Q} | 2 | μL |
| 10× Buffer | 2 | μL |
| H₂O | supplemented to 20 | μL |

After the plasmid is performed with enzyme digestion at 37° C. for 1 hour, it is verified by running the gel, wherein the electrophoresis condition is 90v.

3.6 Deletion of kan Genes by the Second Homologous Recombination

(1) the positive clone after verification in step 3.5 is selected and put into 1 ml LB culture medium containing 30 μg/ml chloromycetin, which is cultured at 220 rpm and 32° C. for 1-2 hours until the bacteria solution becomes cloudy and slight muddy;

(2) adding 1 ml pre-heated LB culture medium containing 30 μg/ml chloromycetin and 2% L-arabinose, which is cultured at 220 rpm and 32° C. for 1 hour;

(3) the solution is immediately transferred to a 42° C. shaking bath at 220 rpm for 30 minutes;

(4) the solution is cultured at 220 rpm and 32° C. for 2-3 hours;

(5) absorbing 1 ml bacteria solution to detect the value of OD600;

(6) when OD600 is ≤0.5, proper amount of the bacteria solution is diluted by 100 times; when OD600 is >0.5, proper amount of bacteria solution is diluted by 1000 times; 5-10 μl bacteria solution diluted by 1:100 is absorbed and coated on the LB solid plate containing 30 μg/ml chloromycetin and 1% L-arabinose;

(7) the solution is cultured at 32° C. for 1-2 days until the size of thallus can be obviously observed.

3.7 Verification of PRV-BAC-CMV-p72-bGH-ΔTK After the Second Homologous Recombination by Extraction Through the Isopropyl Alcohol Precipitation Method

(1) the accurate monocolony in the positive clone in step 3.6 is selected to put into 6 ml LB culture medium containing 30 μg/ml Chl, which is cultured at 32° C. for 24 hours;

The remained steps are same with the step 3.4 and 3.5, which is verified by BamHI enzyme digestion, the enzyme digestion is shown by FIG. 2 and accurate plasmid is selected for sequencing and verification.

3.8 the exterior envelope protein CD2V can be easily integrated onto PRV-BAC-CMV-p72-bGH-ΔTK virus with the above same method to construct virusrPRV-p72-ΔTK-CD2V-ΔgG containing the capsid protein p72 and the exterior envelope protein CD2V.

3.9 the accessory protein B602L can be easily integrated onto rPRV-p72-CD2V-ΔTK-ΔgG virus with the above same method to construct virusrPRV-p72-B602L-CD2V-ΔTK-ΔgG containing the capsid protein p72, the accessory protein B602L and the exterior envelope protein CD2V.

What needs to be clarified is that the integration order of the capsid protein p72, the accessory protein B602L and the exterior envelope protein CD2V genes is reversible and can be arbitrarily adjusted based on requirements and operation habitats.

### Embodiment 4 Saving rPRV-p72-B602L-CD2V-ΔTK-AgG Virus

Purifying the extracted rPRV-p72-B602L-CD2V-ΔTK-ΔgG plasmid and measuring the concentration. Saving this recombinant virus in the BHK-21 cell according to the lipofectamine LTX transfection method. The operation steps are:

\textcircled{1} the plasmid is diluted with OPTI-MEM by adding 2.5 μg plasmid into 125 μL OPTI-MEM and then adding 2.5 μμL plus, which is then mixed uniformly and standing for 5 minutes under room temperature.

\textcircled{2} diluting Lipofectamine LTX: adding 9 μL Lipofectamine LTX into 125 μL OPTI-MEM and then adding 2.5 μL plus, which is mixed slightly and standing for 5 minutes under room temperature.

The mixture in step \textcircled{1} and step \textcircled{2} are mixed uniformly and slightly. The mixture is standing for 5 minutes under room temperature, which is gradually added into a 6-well plate to be uniformly distributed. The 6-well plate is put into a cell incubator of 5% CO₂ at 37° C. for 4-6 hours' culture. Exchanging solution: abandoning the supernatant culture medium and adding 2 mL DMEM/F12 (containing 10% serrum and 1% double antibody). The 6-well plate is put into a cell incubator of 5% CO₂ at 37° C. for culture and the cell state and the formation of virus bacteriophage plaque everyday is observed to obtain rPRV-p72-B602L-CD2V-ΔTK-ΔgG named by the obtained virus.

### Embodiment 5 Detection of p72 and CD2V Protein Expression Through Western Blot

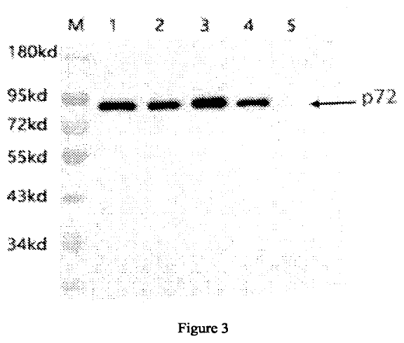

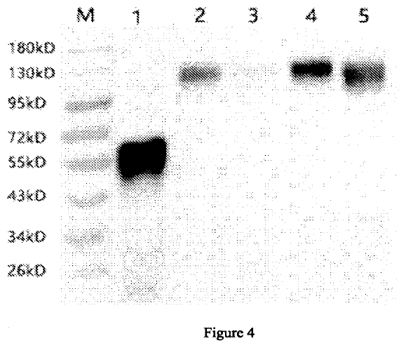

Expanding the propagation of the rPRV-p72-B602L-CD2V-ΔTK-ΔgG virus, centrifuging cell culture mixture at 10000 rpm/min for 5 minutes to collect the supernatant. After processing by loading a buffer, the solution is performed with electrophoresis with 10% SDS-PAGE gel and is transferred onto a PVDF membrane by Wet Transfer (100V, 90 minutes). Then the membrane is blocked with 5% skim milk powder for 2 hours and the anti-flag label mouse monoantibody (for the detection of the capsid protein p72) diluted by 1:4000 times or anti-His label mouse monoantibody (for the detection of the exterior envelope protein CD2V) diluted by 1:4000 times is added. After the solution is reacted under room temperature for 1 hour, it is washed with PBST scrubbing solution for 3 times and added with HRP labeled goat-anti-mouse IgG second antibody diluted by 1:5000 times. The solution is reacted under room temperature for 1 hour and added with the substrate chromogenic solution. After opaque background for 5 minutes, it is observed that a specific band in 70-90kDa has the same size with the predicted protein, i.e. the capsid protein p72 and results are shown as FIG. 3. It is observed that a specific band in 130 kDa has the same results as the predicted ones. As the exterior envelope protein CD2V is a glycosylated protein, it has a larger molecular weight on the SDS-PAGE, as shown by FIG. 4. Results in FIG. 3 and FIG. 4 show that both the capsid protein p72 and the exterior envelope protein CD2V proteins are expressed.

### Embodiment 6 Experiment on Mice Immuned with a Recombinant Virus

8 Balb/c mice (purchased from Zhejiang Chinese Medical University) are divided into two groups randomly and each group has 4 mice, wherein the first group is immuned with the recombinant virus rPRV-p72-B602L-CD2V-ΔTK-ΔgG and the second group is injected with normal saline to act as the blank control Immunity is respectively performed through intramuscular injection and the immunity is performed for 2 times. 21 days after the first immunity, the secondary immunity is performed. 14 days after the secondary immunity, the antibody is detected through ELISA by drawing blood from mice. The specific method is: the elisa plate is respectively coated with recombinant the capsid protein p72 and the exterior envelope protein CD2V, wherein the coating concentration is 0.5 μg/ml and each antigen coats 12 wells (4 wells are added with serum sample of mice after immunity, 4 wells are added with serum of negative mice after immunity, 4 wells are added with confining solution as the control) by 100 μl/well for reaction at 37° C. for 1 hour; the solution is washed with PBST scrubbing solution for 3 times and each time is performed for 5 minutes; goat-anti-mouse IgG labeled by HRP and diluted by 1:5000 is added by 100 μl/well for reaction at 37° C. for 1 hour; the solution is washed with PBST scrubbing solution for 3 times and each time is performed for 5 minutes; the substrate is added by 100 μl/well for coloration and inoculated at 37° C. for 20 minutes. Finally 2M H₂SO₄ is added by 50 μl/well to terminate the reaction. Results are shown the following table:

| | OD450 valude of | OD450 valude of |
|---|---|---|
| Sample | Hunted P72 protein | coated CD2V protein |
| Serum after | 2.105 | 1.878 |
| secondary immunity | | |
| Serum after | 1.984 | 1.763 |
| secondary immunity | | |
| Serum after | 1.853 | 1.696 |
| secondary immunity | | |
| Serum after | 2.532 | 1.954 |
| secondary immunity | | |
| negative serum | 0.232 | 0.12 |
| of blank control | | |
| negative serum | 0.354 | 0.136 |
| of blank control | | |
| negative serum | 0.221 | 0.327 |
| of blank control | | |
| negative serum | 0.246 | 0.225 |
| of blank control | | |
| confining liquid | 0.057 | 0.034 |
| confining liquid | 0.06 | 0.042 |
| confining liquid | 0.083 | 0.075 |
| confining liquid | 0.075 | 0.051 |

Experimental results show that the coated the capsid protein p72 can specifically be bound by antibodies in the serum after the secondary immunity and the average value of OD450 is 2.119; the coated exterior envelope protein CD2V protein can specifically be bound by antibodies in the serum after the secondary immunity and the average value of OD450 is 1.823. It can be obviously seen that all antibodies detected by rPRV-p72-B602L-CD2V-ΔTK-ΔgG have a high concentration, which means that the immunogenicity of the recombinant virusrPRV-p72--B602L-CD2V-ΔTK-ΔgG is excellent and can act as the ASFV-PRV live vector vaccine for researches.

The present application is illustrated by the above embodiments. However it is to be understood that the application is not limited to the specific examples and embodiments described herein. These specific examples and embodiments are included herein to assist those skilled in the art in the practice of the present application. Any person skilled in the art can easily make further improvements and perfections without departing from the spirit and scope of the present invention, so the present invention is only limited by the content and scope of the claims of the present application, which is intended to cover all alternatives and equivalents within the spirit and scope of the application as defined by the appended claims.

1. A recombinant Pseudorabies virus, wherein the genome of the recombinant Pseudorabies virus contains the following exogenous genes:
a nucleotide sequence A coding a capsid protein p72 derived from African swine fever virus or variants thereof, the variants are formed by substitution, deletion or addition of one or more amino acid residues in the amino acid sequence of the capsid protein p72 and have the function of the capsid protein p72;
a nucleotide sequence B coding an accessory protein B602L derived from African swine fever virus and used for prompting the accurate expression and folding of the capsid protein p72 or variants thereof, the variants are formed by substitution, deletion or addition of one or more amino acid residues in the amino acid sequence of the accessory protein and have the function of prompting the accurate expression and-folding, of the capsid protein p72 or variants thereof;
a nucleotide sequence C coding an exterior envelope protein CD2V derived from African swine fever virus or variants thereof, the variants are formed by substitution, deletion or addition of one or more amino acid residues in the amino acid sequence of the exterior envelope protein CD2V and have the function of the exterior envelope protein CD2V.

2. The recombinant Pseudorabies virus according to claim 1, wherein the exogenous genes merely contain the nucleotide sequence A, the nucleotide sequence B and the nucleotide sequence C.

3. The recombinant Pseudorabies virus according to claim 1, wherein the nucleotide sequence A has a homology≥90% to SEQ ID NO:1; the nucleotide sequence B has a homology≥90% to SEQ ID NO:2; the nucleotide sequence C has a homology≥90% to SEQ ID NO:3.

3. The recombinant Pseudorabies virus according to claim 1, wherein the recombinant. Pseudorabies virus is suitable for replicating and expressing the nucleotide sequence A, the nucleotide sequence B and the nucleotide sequence C in cells, the cells are chosen from cells used for virus proliferation.

4. The recombinant Pseudorabies virus according to claim 4, wherein the cells are mammalian cells, bird cells or insect cells.

5. The recombinant Pseudorabies virus according to claim 1, wherein the genome of the recombinant Pseudorabies virus also contains the following exogenous gene:
a nucleotide sequence D coding a capsid protein p49 derived from African swine fever virus or variants thereof, the variants of the capsid protein p49 are formed by substitution, deletion or addition of one or more amino acid residues in the amino acid sequence and have the function of the capsid protein p49.

6. The recombinant Pseudorabies virus according to claim 1, wherein a deletion and/or replacement occurs in at least one of non-essential replication regions in the genome and the non-essential replication regions are chosen from more than one of gC, gE, gG, gI, gM, TK, RR, PK coding regions of Pseudorabies virus.

7. A method, of constructing the recombinant Pseudorabies virus according to claim 1 comprising the following steps:
S1 substituting the coding region TK genes in a genome of Pseudorabies viral. HL strain PRV-BAC with a codon-optimized gene expression cassette expressing the capsid protein p72 derived from African swine fever virus to obtain PRV-BAC-p72-ΔTK;
S2 inserting a codon-optimized gene expression cassette of the accessory protein B602L derived from African swine fever virus into the capsid protein p72 genes to obtain PRV-BAC-p 72-B 602L-ΔTK;
S3 substituting the coding region gG genes of the PRV-BAC-p72-B602L-ΔTK obtained in step (2) with a codon-optimized gene expression cassette of the exterior envelope protein CD2V derived from African swine fever virus to obtain PRV-BAC-p72-B602L-CD2V-ΔTK-ΔgG;
S4 transfecting Hamster kidney fibroblast cells with the PRV-BAC-p72-B602L -CD2V-ΔTK-ΔgG obtained in step (3) to rescue and obtain the recombinant Pseudorabies virus that simultaneously expresses the capsid protein p72, the accessory protein B602L and the exterior envelope protein CD2V and is named as rPRV-p72-B602L-CD2V-ΔTK-ΔgG.

8. An African swine fever vaccine, wherein the African swine fever vaccine at least contains the recombinant Pseudorabies virus according to claim 1 as an immunogen.

9. The African swine fever vaccine according to claim 9, wherein the recombinant Pseudorabies virus as an immunogen is the recombinant Pseudorabies virus according to claim 2.

10. The African swine fever vaccine according to claim 9, wherein the recombinant Pseudorabies virus as an immunogen is the recombinant Pseudorabies virus according to claim 3.

11. The African swine fever vaccine according to claim 9, wherein the recombinant Pseudorabies virus as an immunogen is the recombinant Pseudorabies virus according to claim 3.

12. The African swine fever vaccine according to claim 9, wherein the recombinant Pseudorabies virus as an, immunogen is the recombinant Pseudorabies virus according to claim 4.

13. The African swine fever vaccine according to claim 9, wherein the recombinant Pseudorabies virus as an immunogen is the recombinant Pseudorabies virus according to claim 5.

14. The African swine fever vaccine according to claim 9, wherein the recombinant Pseudorabies virus as an, immunogen is the recombinant Pseudorabies virus according to claim 6.

15. The African swine fever vaccine according to claim 9, wherein the recombinant Pseudorabies virus as an immunogen is the recombinant Pseudorabies virus according to claim 7.


SEQUENCE LISTING (8 sequences)

SEQ ID NO: 1 - DNA, 1938 bp, synthetic construct
  source 1..1938: mol_type = other DNA
atggcttctggcggcgctttttgcctgatcgccaacgacggcaaggctgataagatcatcctggctcaggacctgctgaactcccggatcagcaatatcaagaatgtgaacaagagctacggcaagccagatcccgagcctaccctgtctcagatcgaggagacacacctggtgcatttcaatgcccacttcaagccatacgtgcccgtgggcttcgagtataacaaggtgagaccccacaccggcacacctaccctgggcaataagctgacctttggcatcccacagtatggcgacttctttcatgatatggtgggccaccatatcctgggagcttgtcactccagctggcaggacgctccaatccagggcaccagccagatgggcgctcatggccagctgcagacattcccacgcaacggctacgactgggataatcagacccccctggagggcgccgtgtatacactggtggatccttttggcagacctatcgtgccaggcaccaagaacgcttaccgcaatctggtgtactattgcgagtaccccggcgagagactgtatgagaacgtgcgcttcgacgtgaatggcaactctctggacgagtactcttccgatgtgaccacactggtgagaaagttttgtatccccggcgataagatgaccggctataagcacctggtgggacaggaggtgagcgtggagggcacatctggccctctgctgtgcaatatccacgacctgcataagccacaccagtctaagcccatcctgaccgacgagaacgatacacagcgcacctgttcccatacaaatccaaagttcctgagccagcattttcccgagaactctcacaatatccagaccgccggcaagcaggatatcacacctatcaccgacgctacatacctggatatcaggcggaacgtgcactattcttgcaatggcccccagacacctaagtactatcagccccctctggccctgtggatcaagctgaggttctggtttaatgagaacgtgaatctggctatcccttccgtgtccatcccattcggcgagcggtttatcaccatcaagctggcctcccagaaggacctggtgaacgagttcccaggcctgtttgtgaggcagtcccggttcatcgctggcaggcccagcagacgcaacatccggttcaagccttggtttatcccaggcgtgatcaatgagatctccctgaccaacaatgagctgtacatcaacaatctgtttgtgacacctgagatccacaacctgttcgtgaagagggtgcggtttagcctgatccgggtgcataagacccaggtgacacacaccaacaataaccaccatgacgagaagctgatgagcgccctgaagtggccaatcgagtatatgttcatcggcctgaagcctacatggaacatctctgaccagaatccacaccagcatagagattggcataagttcggccacgtggtgaacgccatcatgcagcctacccaccatgctgagatcagctttcaggaccgcgatacagccctgccagacgcttgcagctctatctctgatatctcccccgtgacctaccctatcacactgccaatcatcaagaacatctccgtgaccgcccatggcatcaatctgatcgacaagttccccagcaagttttgttccagctacatccctttccactatggcggcaacgctatcaagaccccagacgatcccggcgccatgatgatcacatttgctctgaagcccagggaggagtaccagccttccggccacatcaatgtgagcagagcccgcgagttctacatctcttgggacaccgattatgtgggctccatcaccacagctgatctggtggtgagcgcctctgctatcaactttctgctgctgcagaatggctctgccgtgctgcggtattccaca

SEQ ID NO: 2 - DNA, 1590 bp, synthetic construct
  source 1..1590: mol_type = other DNA
atggccgagtttaacatcgacgagctgctgaagaatgtgctggaggatccatctaccgagatctccgaggagacactgaagcagctgtaccagcggaccaacccctataagcagttcaagaatgacagcagagtggctttctgctcttttaccaacctgcgcgagcagtacatcaggcggctgatcatgacatccttcatcggctacgtgttcaaggccctgcaggagtggatgccaagctactctaagcccacccacaccacaaagacactgctgagcgagctgatcacactggtggacaccctgaagcaggagaccaacgatgtgccctccgagagcgtggtgaatacaatcctgagcatcgctgattcttgcaagacccagacacagaagtctaaggaggccaagaccacaatcgactcctttctgagggagcacttcgtgtttgatcctaacctgcatgctcagtctgcctacacatgtgctgacaccaacgtggatacatgcgcttccatgtgcgccgacaccaatgtggatacatgcgcttctatgtgcgctgatactaacgtggacacctgcgcttccacctgtacaagcaccgagtataccgacctggccgatcccgagagaatccctctgcacatcatgcagaagacactgaacgtgcccaatgagctgcaggctgacatcgatgccatcacacagacccctcagggatacagggctgctgctcatatcctgcagaatatcgagctgcaccagagcatcaagcatatgctggagaaccctagggccttcaagccaatcctgtttaatacaaagatcacccggtatctgtctcagcacatcccccctcaggacaccttctacaagtggaactactatatcgaggataattatgaggagctgagggctgccacagagtctatctatccagagaagcccgacctggagttcgcttttatcatctacgacgtggtggattccagcaaccagcagaaggtggacgagttctactataagtacaaggatcagatcttttctgaggtgtcttccatccagctgggcaactggaccctgctgggctccttcaaggccaatagagagaggtacaactacttcaaccagaacaatgagatcatcaagaggatcctggaccggcacgaggaggatctgaagatcggcaaggagatcctgagaaacaccatctatcataagaaggctaagaatatccaggagacaggacctgacgctccaggactgtctatctacaattccacattccataccgattccggcatcaagggcctgctgagctttaaggagctgaagaacctggagaaggcttccggcaatatcaagaaggccagagagtacgactttatcgacgattgtgaggagaagatcaagcagctgctgagcaaggagaacctgacccctgacgaggagtctgagctgatcaagacaaagaagcagctggataacgctctggagatgctgaatgtgccagacgataccatccgcgtggatatgtgggtgaacaataacaataagctggagaaggagatcctgtataccaaggccgagctg

SEQ ID NO: 3 - DNA, 1011 bp, synthetic construct
  source 1..1011: mol_type = other DNA
atgatcatcctgatcttcctgatcttttctaacatcgtgctgtccatcgactactgggtgtctttcaataagacaatcatcctggattccaacatcaccaatgacaacaatgatatcaacggcgtgtcctggaatttctttaacaatagcttcaacaccctggccacatgcggcaaggctggcaacttttgcgagtgttctaattactctacctccatctataacatcacaaacaattgttccctgaccatcttcccacacaatgacgtgtttgataccacataccaggtggtgtggaaccagatcatcaattatacaatcaagctgctgacccctgccacaccccctaacatcacctacaactgcacaaattttctgatcacctgtaagaagaacaatggcaccaacacaaatatctatctgaacatcaatgacaccttcgtgaagtacacaaatgagagcatcctggagtacaactggaacaactctaacatcaacaacttcaccgctacatgcatcatcaacaataccatcagcacatctaacgagaccacactgatcaattgtacctacctgacactgtccagcaactacttctataccttctttaagctgtacagcctgaggaagcggaagaagcacgtggaggagatcgagtccccaccccctgagagcaacgaggaggagcagtgccagcacgacgataccacatctatccatgagccctcccctagagagcccctgctgccaaagccctacagccgctaccagtataacacacctatctactatatgaggccatctacccagcccctgaatcctttcccactgcccaagccttgcccaccccctaagccttgtccaccccctaagccatgcccaccacctaagccatgtccatccgccgagtcctatagcccacccaagcctctgccaagcatccccctgctgcctaacatccctccactgagcacccagaatatctctctgatccatgtggatagaatcatc

SEQ ID NO: 4 - DNA, 79 bp, synthetic construct
  source 1..79: mol_type = other DNA
gttcgtagaagcggttgtggcagcggatccccgcccggaagcgcgccggggacattgattattgactagttattaatag

SEQ ID NO: 5 - DNA, 68 bp, synthetic construct
  source 1..68: mol_type = other DNA
tacacatggctttatacgcgccccgagccccctcccacgccgtcgtcgttagacatgaccatagagcc

SEQ ID NO: 6 - protein, 646 aa, African swine fever virus
MASGGAFCLIANDGKADKIILAQDLLNSRISNIKNVNKSYGKPDPEPTLSQIEETHLVHFNAHFKPYVPVGFEYNKVRPHTGTPTLGNKLTFGIPQYGDFFHDMVGHHILGACHSSWQDAPIQGTSQMGAHGQLQTFPRNGYDWDNQTPLEGAVYTLVDPFGRPIVPGTKNAYRNLVYYCEYPGERLYENVRFDVNGNSLDEYSSDVTTLVRKFCIPGDKMTGYKHLVGQEVSVEGTSGPLLCNIHDLHKPHQSKPILTDENDTQRTCSHTNPKFLSQHFPENSHNIQTAGKQDITPITDATYLDIRRNVHYSCNGPQTPKYYQPPLALWIKLRFWFNENVNLAIPSVSIPFGERFITIKLASQKDLVNEFPGLFVRQSRFIAGRPSRRNIRFKPWFIPGVINEISLTNNELYINNLFVTPEIHNLFVKRVRFSLIRVHKTQVTHTNNNHHDEKLMSALKWPIEYMFIGLKPTWNISDQNPHQHRDWHKFGHVVNAIMQPTHHAEISFQDRDTALPDACSSISDISPVTYPITLPIIKNISVTAHGINLIDKFPSKFCSSYIPFHYGGNAIKTPDDPGAMMITFALKPREEYQPSGHINVSRAREFYISWDTDYVGSITTADLVVSASAINFLLLQNGSAVLRYST

SEQ ID NO: 7 - protein, 530 aa, African swine fever virus
MAEFNIDELLKNVLEDPSTEISEETLKQLYQRTNPYKQFKNDSRVAFCSFTNLREQYIRRLIMTSFIGYVFKALQEWMPSYSKPTHTTKTLLSELITLVDTLKQETNDVPSESVVNTILSIADSCKTQTQKSKEAKTTIDSFLREHFVFDPNLHAQSAYTCADTNVDTCASMCADTNVDTCASMCADTNVDTCASTCTSTEYTDLADPERIPLHIMQKTLNVPNELQADIDAITQTPQGYRAAAHILQNIELHQSIKHMLENPRAFKPILFNTKITRYLSQHIPPQDTFYKWNYYIEDNYEELRAATESIYPEKPDLEFAFIIYDVVDSSNQQKVDEFYYKYKDQIFSEVSSIQLGNWTLLGSFKANRERYNYFNQNNEIIKRILDRHEEDLKIGKEILRNTIYHKKAKNIQETGPDAPGLSIYNSTFHTDSGIKGLLSFKELKNLEKASGNIKKAREYDFIDDCEEKIKQLLSKENLTPDEESELIKTKKQLDNALEMLNVPDDTIRVDMWVNNNNKLEKEILYTKAEL

SEQ ID NO: 8 - protein, 337 aa, African swine fever virus
MIILIFLIFSNIVLSIDYWVSFNKTIILDSNITNDNNDINGVSWNFFNNSFNTLATCGKAGNFCECSNYSTSIYNITNNCSLTIFPHNDVFDTTYQVVWNQIINYTIKLLTPATPPNITYNCTNFLITCKKNNGTNTNIYLNINDTFVKYTNESILEYNWNNSNINNFTATCIINNTISTSNETTLINCTYLTLSSNYFYTFFKLYSLRKRKKHVEEIESPPPESNEEEQCQHDDTTSIHEPSPREPLLPKPYSRYQYNTPIYYMRPSTQPLNPFPLPKPCPPPKPCPPPKPCPPPKPCPSAESYSPPKPLPSIPLLPNIPPLSTQNISLIHVDRII